(19) United States
(12) Patent Application Publication
(10) Pub. No.: US 2006/0264652 A1
(43) Pub. Date: Nov. 23, 2006

(54) Process for preparing 4-chloro-3-hydroxybutanoic acid ester

(76) Inventors: Sung-Wook Cho, Daejeon (KR); Jay-Hyok Chang, Daejon (KR); Kyu-Woong Lee, Daejeon (KR); Ki-Kon Lee, Daejeon (KR); Byung-Ran So, Daejeon (KR); Hyun-Ik Shin, Daejeon (KR)

Correspondence Address: SUGHRUE MION, PLLC, 2100 PENNSYLVANIA AVENUE, N.W., SUITE 800, WASHINGTON, DC, 20037, US

(21) Appl. No.: 10/553,170

(22) PCT Filed: Apr. 14, 2004
(86) PCT No.: PCT/KR04/00869

§ 371 Date: Oct. 17, 2005

(30) Foreign Application Priority Data
Apr. 16, 2003 (KR) 10-2003-0023968

(51) Int. Cl. C07C 253/02

(52) U.S. Cl. 558/347; 562/579

(57) ABSTRACT

The present invention relates to a process for preparing 4-chloro-3-hydroxybutanoic acid ester, an intermediate for preparing atorvastatin, in high purity and yield, by comprising the steps of 1) reacting epichlorohydrin of formula (2) with cyanide of formula (3) under the condition of pH ranging from 7 to 8, to form the 4-chloro-3-hydroxybutyronitrile of formula (4) and 2a) dissolving the 4-chloro-3-hydroxybutyronitrile of formula (4) in an alcoholic solvent and reacting it with hydrogen chloride, or 2b) reacting the 4-chloro-3-hydroxybutyronitrile of formula (4) in an alcoholic solvent saturated with hydrogen chloride, to form the 4-chloro-3-hydroxybutyronitrile acid ester of formula (I).

### TECHNICAL FIELD

The present invention relates to a process for preparing 4-chloro-3-hydroxybutanoic acid ester. More specifically, the present invention relates to a process for preparing 4-chloro-3-hydroxybutanoic acid ester of high optical and chemical purity in high yield through the optimization of the reaction pH, addition order of reactants, and/or amounts, etc. of reaction solvent and the reactants.

### BACKGROUND ART

4-Chloro-3-hydroxybutanoic acid ester of the following formula:

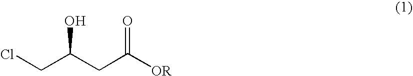

, wherein R is C₁₋₄alkyl,

is a useful intermediate for preparing atorvastatin, a therapeutic agent of hyper-lipidemia.

A process for preparing the above 4chloro-3-hydroxybutanoic acid ester, known in the art, comprises the following steps of:

1) reacting epichlorohydrin of the following formula:

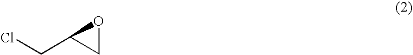

with a cyanide of the following formula:
M(CN)ₙ (3)

, wherein M is a cation, and n is an integer of 1 to 3,

to form 4-chloro-3-hydroxybutyronitrile of the following formula:

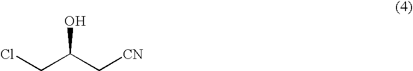

2) subjecting the 4-chloro-3-hydroxybutyronitrile of the above formula (4) to acid hydrolysis to form the 4-chloro-3-hydroxybutanoic acid ester of the above formula (1).

The above process may be depicted by the following reaction scheme:

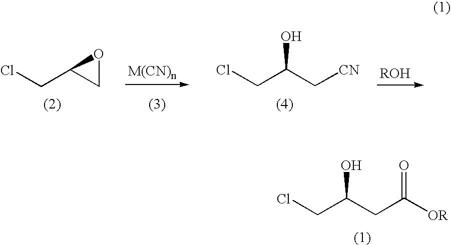

First, some processes to prepare 4-chloro-3-hydroxybutyronitrile in step 1) are known in the art: reacting chiral epichlorohydrin with liquid hydrogen cyanide under heating in a sealed container for several days [Hormann, Ber., 1879, 12, 23], employing hydrogen cyanide with potassium cyanide as a catalyst [F. Binon, Bull. Soc. Chim. Belges., 1963, 72, 166], performing the reaction under the neutral condition by simultaneously introducing a mixed aqueous solution of sodium cyanide and potassium cyanide with an aqueous solution of acetic acid [Culvenor, J. Chem. Soc., 1950, 3123], etc.

However, the Hormann's method employing liquid hydrogen cyanide is not suitable for commercial production because liquid hydrogen cyanide is very dangerous to handle, and it requires extremely long reaction time and a specially designed pressure-resistant container for industrial use. The Binon's method also has the same problem of using hydrogen cyanide. Also, the Culvenor's method has difficulty to control the speed of simultaneous introduction of an aqueous metal cyanide solution with an acid solution to maintain the optimal pH.

In order to resolve the above-mentioned problems and to provide an economical process suitable for large-scale industrial production, various improved processes have been developed. For example, Japanese Patent No. 5310671 by Daiso Co., Ltd. in Japan discloses a process characterized by maintaining the reaction pH within the basic range of 8 to 10 by simultaneously introducing an inorganic acid solution and an aqueous solution of alkali metal cyanide into an aqueous solution of epichlorohydrin. This process tried to resolve such problems as formation of the side products of 3-hydroxyglutaronitrile and 4-hydroxycrotonitrile under basic pH and elevated temperature, as described in Org. Syntheses, CV 5, 614. However, it is not so easy to adjust pH by simultaneously introducing sulfuric acid solution and basic aqueous cyanide solution into the epichlorohydrin solution, and particularly, the heat of neutralization occurred from simultaneous introducing an acid and a base may be a concern in terms of the control of the reaction temperature.

Subsequently, a process to prepare 4-chloro-3-hydroxybutanoic acid ester in step 2) comprises the steps of subjecting 4-chloro-3-hydroxybutyronitrile to hydrolysis under aqueous acidic conditions to form a carboxylic acid (4-chloro-3-hydroxybutanoic acid), which was further transformed to 4-chloro-3-hydroxybutanoic acid ester. This process may be depicted by the following reaction scheme:

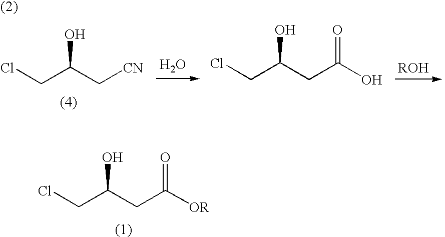

In the above reaction, R—C(OH)═NH is formed as an intermediate, and hydrolysis of the imine (═NH) forms a carboxylic acid. The reaction is a conventional hydrolysis employing an aqueous acid solution, and has such problems that it should be performed in the reflux temperature, and often stops in the amide intermediate which can hardly be hydrolyzed.

Another known process (Finner's reaction) comprises the steps of dissolving 4-chloro-3-hydroxybutyronitrile in an alcohol or a mixed solution of an alcohol and an inert solvent, performing the reaction at a low temperature for a long time with blowing hydrogen chloride gas thereto to form an imidate as an intermediate, and hydrolyzing the imidate with an aqueous acid solution. The above process may be depicted by the following reaction scheme:

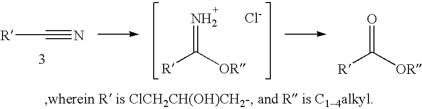

According to the process described in a literature by Geza Braun, J. Amer. Chem. Soc., 1930, 52, 3167, the reactants are cooled down in a mixed solution of ethanol and ethyl ether, the reaction is performed with an extreme excess of hydrogen chloride gas over several hours, and the reaction mixture is concentrated and the residual hydrogen chloride gas is removed through distilling the solvent therefrom. An imidate compound obtained from the above reaction is dissolved in water again, and hydrolyzed to obtain the desired ester compound. In this case, if the excessive hydrogen chloride is not removed, a carboxylic acid is formed as a byproduct with the ethyl ester, and thus, the concentration should be performed as completely as possible when distilling the solvent under reduced pressure. For industrial application, the above process has several problems such that an anti-rust reactor should be very carefully selected due to the presence of excessive hydrogen chloride and its productivity is very low due to an extremely long reaction time. In addition, the present inventors performed the reaction according to the above literature, and as a result, confirmed that the reaction has such inconveniences that an impurity with unknown structure is formed, and so the desired product of high purity can be obtained only after a purification process such as distillation, and the reaction takes a long time of several days.

Therefore, in order to resolve the above problems and to provide an economical process suitable for large scale industrial production, various improved processes have been developed. For example, Japanese Patent No. 04124157 discloses a process for preparing 4-chloro-3-hydroxybutanoic acid ester of high optical activity. This process provides 4-chloro-3-hydroxybutanoic acid ester with high optical activity by heating 4-chloro-3-hydroxybutyronitrile in a concentrated hydrochloric acid solution, extracting the solution to obtain 4-chloro-3-hydroxybutanoic acid, and esterifying the isolated carboxylic acid with a small amount of an acid catalyst in an alcoholic solvent. According to the patent, 4-hydroxy-3-hydroxybutyronitrile is treated with concentrated hydrochloric acid and heated to obtain an aqueous solution of 4-chloro-3-hydroxybutanoic acid. The resulting aqueous solution is concentrated under reduced pressure and extracted with a solvent. The extract concentrate is purified with a column chromatography, and then, reacted with a suitable alcohol under an acid catalysis to afford 4-chloro-3-hydroxybutanoic acid ester. However, this process is not suitable for practical application, either, in that the employment of an extremely excessive amount of concentrated hydrochloric acid followed by concentration under reduced pressure may cause corrosion of apparatus. Moreover, the concentration of water employed as a reaction solvent under reduced pressure is not easy and further, several-times of repeated extractions of 4-chloro-3-hydroxybutanoic acid are required due to its good solubility into an aqueous phase.

### DISCLOSURE OF THE INVENTION

The present inventors have performed extensive studies to resolve the above described problems of the prior arts. As a result, the present inventors found a certain optimal range of the reaction pH. The inventors also found that the desired product with high optical activity can be obtained in high purity and yield by switching the order of addition of reactants, and/or modifying kinds, amounts, etc. of a reaction solvent and the reactants.

Therefore, the purpose of the present invention is to provide a process that can prepare 4-chloro-3-hydroxybutanoic acid ester of high optical activity and purity in good yield, low cost, and high suitability for large scale operation.

One aspect of the present invention provides a process for preparing 4-chloro-3-hydroxybutyronitrile of formula:

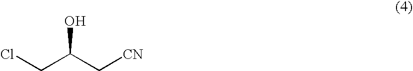

, comprising the step of

1) reacting epichlorohydrin of formula:

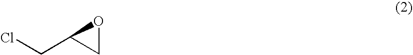

with a cyanide of formula:
M(CN)ₙ (3)

, wherein M is a cation, and n is an integer of 1 to 3,

under the pH condition ranging from 7 to 8, particularly from 7.3 to 7.8, to form the 4-chloro-3-hydroxybutyronitrile of formula (4).

A second aspect of the present invention provides a process for preparing 4-chloro-3-hydroxybutanoic acid ester of formula:

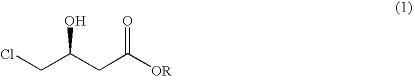

, wherein R is C₁₋₄alkyl,

comprising the step of

2a) dissolving 4-chloro-3-hydroxybutyronitrile of formula (4) in an alcoholic solvent, and then, reacting it with hydrogen chloride, or

2b) reacting the 4-chloro-3-hydroxybutyronitrile of formula (4) in an alcoholic solvent saturated with hydrogen chloride,

to form the 4-chloro-3-hydroxybutanoic acid ester of formula (1).

A third aspect of the present invention provides a process for preparing 4-chloro-3-hydroxybutanoic acid ester of formula (1) comprising the above step 1) and step 2a) or 2b).

Hereinafter, the present invention will be explained in detail.

### 1) Step 1): Preparation of 4chloro-3-hydroxybutyronitrile

The present inventors found that the composition of the reaction product varies depending on the pH at which epichlorohydrin reacts with cyanide, as depicted in the following reaction scheme:

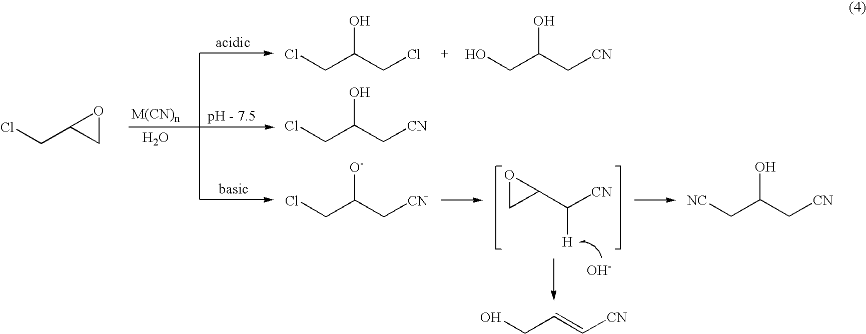

First, when the reaction solution is acidic, the ring-opening reaction of epichlorohydrin is accelerated by acid catalysis, to form considerable amounts of 3,4-dihydroxybutyronitrile and 1,3-dichloroisopropanol, and their amounts increase as the acidity becomes stronger.

Second, when the reaction solution is basic, the epoxy ring is attacked by cyanide, and thus, the desired 4-cyano-3-hydroxybutyronitrile is produced as a main product, but hydroxyl anion formed during the reaction attacks the chloromethyl group intramolecularly to form another epoxy ring resulting in 3,4-epoxybutyronitrile, which is attacked again by cyanide group to form 3-hydroxyglutaronitrile. Alternatively, β-elimination reaction of 3,4-epoxybutyronitrile by the action of base forms 4-hydroxycrotononitrile.

Therefore, as discovered by Daiso Co., Ltd., the present inventors confirmed that it is very important to adjust the pH of the reaction solution. However, while Daiso Co., Ltd, reported that the pH in the range of 8 to 10 is the most preferable, the present inventors newly found that the formation of byproducts can be minimized and the reaction can be performed most efficiently by adjusting the pH of the reaction solution to the range of 7 to 8, particularly 7.3 to 7.8. Moreover, since it is not easy to simultaneously introduce the two reactants, one of which is acidic and the other is basic, with delicately maintaining the reaction pH within a certain range, the present inventors developed a process that can very strictly control the conditions of the reaction, by switching the order of addition of the reactants in step 1).

Specifically, in the present invention, metal cyanide and an inorganic acid are introduced into a reactor and the pH is adjusted to the desired range. Subsequently, epichlorohydrin is added thereto to carry out the reaction under the condition in which the pH is controlled in a relatively simple manner. That is, the pH of the reaction solution is adjusted to 7.0 to 8.0, preferably 7.3 to 7.8, and then, epichlorohydrin is added thereto dropwise.

The kinds of metal cyanide used for the above process include an alkali metal cyanide such as sodium cyanide, potassium cyanide, etc., calcium cyanide, barium cyanide and the like, but sodium cyanide and potassium cyanide are particularly preferable because they are readily available and have been widely used in the industry. The kinds of inorganic acid introduced for adjusting the pH include hydrochloric acid, nitric acid, sulfuric acid, sulfonic acid, phosphoric acid, methanesulfonic acid, etc. Preferable are sulfonic acid, sulfuric acid and hydrochloric acid.

The reaction with the inorganic acid may be preformed in a mixture of alcohol and water, or water, and preferably, in water, and water may be used in the weight ratio of 2 to 20 based on the weight of epichlorohydrin. However, considering stirring efficiency and economical aspect, it is preferable to use water in the weight ratio of 3 to 6, more preferably 3 to 4. The reaction temperature may be in the range of 0 to 90° C., but the temperature range of 10 to 40° C. is preferable to maintain reasonable reaction rate, and to suppress the formation of byproducts. Particularly, the temperature range of 15 to 25° C. is the most preferable.

Upon completion of the reaction, salt compound formed therefrom may be filtered depending on the kinds of metal cyanide and acid introduced into the reaction solution, and the filtrate is extracted with an organic solvent, and the extract is concentrated to obtain the desired 4-chloro-3-hydroxybutyronitrile. The suitable kinds of extraction solvent include toluene, butanol, ethyl acetate, butyl acetate, dichloromethane, etc. In terms of extracting capacity, ethyl acetate, butyl acetate, butanol, dichloromethane, etc. are preferable, and ethyl acetate and dichloromethane are more preferable.

### 2) Step 2): Preparation of 4-chloro-3-hydroxybutanoic acid ester

In this step, the present inventors tried to employ minimal amount of acid and to omit a step of extracting 4-chloro-3-hydroxybutanoic acid as an intermediate, and simultaneously, to obtain the desired product in high purity and yield for a shortened period of time. As a result, the present inventors found that the desired carboxylic acid ester can be rapidly prepared in high purity by dissolving 4-chloro-3-hydroxybutyronitrile in an alcoholic solvent and bubbling hydrogen chloride gas thereto. Also, the same reaction profiles could be obtained by using an alcoholic solvent preliminarily saturated with hydrogen chloride gas.

The alcoholic solvent used in this step may be C₁₋₄alcohol. It may be used alone, or used in combination with another solvent. In that case, diethyl ether or diisopropyl ether is preferable as co-solvent. Most preferably, the alcoholic solvent is used alone. The weight-by-weight ratio of the alcohol to 4-chloro-3-hydroxybutyronitrile may be in the range of 1 to 10, preferably 1.5 to 4, more preferably 1.5 to 2.5, in terms of economical efficiency and reaction rate.

The amount of hydrogen chloride may be in the range of 1 to 10 mole equivalents, preferably 1 to 6 mole equivalents, for a fast reaction and work-up of the residual hydrogen chloride. The reaction temperature may be in the range of 0 to 80° C., preferably 15 to 50° C., more preferably 15 to 25° C., considering the purity of reaction. In case that optically active epichlorohydrin is used as the starting material, 4-chloro-3-hydroxybutanoic acid ester obtained from the above reaction retains the optical purity.

In addition, upon completion of the reaction, the present invention has the advantage to increase the productivity by reducing the steps of reaction through using relatively very small amount of alcoholic solvent which enables direct extraction with an organic solvent without concentration of alcoholic solvent, while excess alcoholic solvent was distilled under reduced pressure in the prior art.

### BEST MODE FOR CARRYING OUT THE INVENTION

The present invention will be more specifically illustrated by the following examples. However, the following examples should not be construed as limiting the scope of the present invention in any way.

### EXAMPLE 1

### Preparation of 4-chloro-3-hydroxybutyronitrile (NaCN/H₂ SO₄)

Sodium cyanide (9.93 g) was dissolved in 60 ml of distilled water, and the solution was cooled down in ice bath. To this solution was added dropwise sulfuric acid of 9.87 g while maintaining the temperature to 20° C. or lower, and the pH was measured and confirmed to be 7.7. To the above solution was added 15 g of epichlorohydrin, and then, the mixture was stirred at room temperature. Upon completing the reaction, the reaction solution was extracted three times with ethyl acetate, and concentrated under reduced pressure to obtain 17.2 g (yield: 89%) of the title compound as deep yellow oil. Chemical purity (GC): 96.5%

¹H-NMR (CDCl₃) δ 4.21 (1H, m), 3.66 (2H, d, J=5.6 Hz), 3.03 (1H, d, J=5.6 Hz, —OH), 2.73 (2H, m)

¹³C-NMR (CDCl₃) δ 117.1, 67.3, 47.3, 23.3

### EXAMPLE 2

### Preparation of 4-chloro-3-hydroxybutyronitrile (KCN/H₂ SO₄)

The title compound of 17.8 g (yield: 92%) was obtained according to substantially the same method as in Example 1 except using potassium cyanide instead of sodium cyanide. Chemical purity (GC): 96.7%

### EXAMPLE 3

### Preparation of 4-chloro-3-hydroxybutyronitrile (KCN/HCl)

The title compound of 17.4 g (yield: 90%) was obtained according to substantially the same method as in Example 1 except using potassium cyanide instead of sodium cyanide and concentrated hydrochloric acid instead of sulfuric acid. Chemical purity (GC): 95.8%

### EXAMPLE 4

### Preparation of 4-chloro-3(S)-hydroxybutyronitrile (KCN/H₂SO₄)

The title compound of 17.6 g (yield: 91%) was obtained according to substantially the same method as in Example 1 except using potassium cyanide instead of sodium cyanide and (S)-epichlorohydrin as epichlorohydrin. Chemical purity (GC): 96.5%; Optical purity (HPLC): 99.2% ee

### EXAMPLE 5

### Preparation of 4-chloro-3-hydroxybutanoic acid ethyl ester

Ethanol was cooled down, and anhydrous hydrogen chloride gas was bubbled slowly thereto. The obtained solution was titrated to prepare 10 N ethanol solution of hydrogen chloride. The ethanol solution of hydrogen chloride of 30 ml was mixed with 11.96 g of 4chloro-3-hydroxybutyronitrile, and the reaction was performed while heating to 60° C. under nitrogen atmosphere. Upon completing the reaction, the reaction solution was cooled down, and extracted with 30 ml of distilled water and 50 ml of ethyl acetate, and the aqueous phase was further extracted twice with 50 ml of ethyl acetate. The extract was collected and concentrated under reduced pressure to obtain the title compound of 15.5 g (yield: 93%). Chemica purity (GC): 96.8%

¹H-NMR (CDCl₃) δ 4.20˜4.30 (1H, m), 4.18 (2H, q, J=7.3 Hz), 3.55˜3.65 (2H, m), 3.17 (1H, br), 2.55˜2.70 (2H, m), 1.28 (3H, t, J=7.3 Hz)

¹³C-NMR (CDCl₃) δ 171.8, 68.0, 61.0, 48.2, 38.5, 14.1

### EXAMPLE 6

### Preparation of 4-chloro-3-hydroxybutanoic acid methyl ester

The title compound of 15.8 g (yield: 95%) was obtained according to substantially the same method as in Example 1 except using 4-chloro-3(S)-hydroxybutyronitrile as 4-chloro-3-hydroxybutyronitrile and methanol instead of ethanol. Chemical purity (GC): 97.1%; Optical purity (HPLC): 99.2% ee

¹H-NMR (CDCl₃) δ 4.28 (1H, m), 3.70 (3H, s), 3.61 (2H, m), 3.40 (1H, br), 2.65 (2H, m)

¹³C-NMR (CDCl₃) δ 172.2, 68.0, 52.0, 38.2, 38.8

### INDUSTRIAL APPLICABILITY

According to the present invention, 4-chloro-3-hydroxybutyronitrile of high purity can be obtained in high yield by reacting epichlorohydrin with cyanide at the pH range of 7 to 8, particularly, 7.3 to 7.8, preferably by adjusting the pH to the above range by preliminarily mixing aqueous metal cyanide with an inorganic acid at room temperature and room pressure, and then, adding epichlorohydrin thereto to perform the reaction. Also, 4-chloro-3-hydroxybutyronitrile with high optical activity can be obtained with using chiral epichlorohydrin. Moreover, 4-chloro-3-hydroxybutanoic acid ester can be prepared on a large scale in high purity and yield through one-step reaction from 4-chloro-3-hydroxybutyronitrile. Further, from 4-chloro-3-hydroxybutyronitrile with optical activity, 4-chloro-3-hydroxybutanoic acid ester retaining the optical activity can be obtained in high yield and purity.

1. A process for preparing 4-chloro-3-hydroxybutyronitrile of formula: 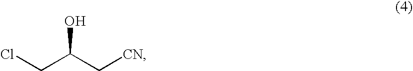
comprising the step of
1) reacting epichlorohydrin of formula: 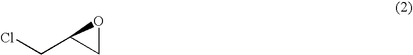
with a cyanide of formula:
M(CN)ₙ (3),
wherein M is a cation, and n is an integer of 1 to 3,
under the condition of pH ranging from 7 to 8, to form the 4-chloro-3-hydroxybutyronitrile of formula (4).

2. A process for preparing 4-chloro-3-hydroxybutanoic acid ester of formula: 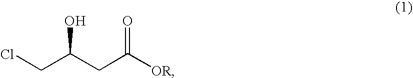
wherein R is C₁₋₄alkyl,
comprising the step of
2a) dissolving 4-chloro-3-hydroxybutyronitrile of formula: 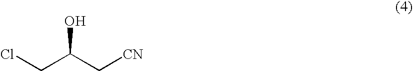
in an alcoholic solvent, and then, reacting it with hydrogen chloride, or
2b) reacting the 4-chloro-3-hydroxybutyronitrile of formula (4) in an alcoholic solvent saturated with hydrogen chloride,
to form the 4-chloro-3-hydroxybutanoic acid ester of formula (1).

3. A process for preparing 4-chloro-3-hydroxybutanoic acid ester of formula: 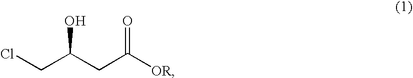
wherein R is C₁₋₄alkyl,
comprising the step of
2a) dissolving 4-chloro-3-hydroxybutyronitrile of formula: 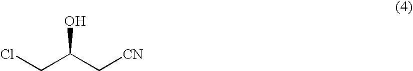
in an alcoholic solvent, and then, reacting it with hydrogen chloride, or
2b) reacting the 4-chloro-3-hydroxybutyronitrile of formula (4) in an alcoholic solvent saturated with hydrogen chloride,
to form the 4-chloro-3-hydroxybutanoic acid ester of formula (1),
comprising the steps of:
1) reacting epichlorohydrin of formula: 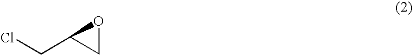
with a cyanide of formula:
M(CN)ₙ (3),
wherein M is a cation, and n is an integer of 1 to 3,
under the condition of pH ranging from 7 to 8, to form the 4-chloro-3-hydroxybutyronitrile of formula (4),
under the condition of pH ranging from 7 to 8, to form 4-chloro-3-hydroxybutyronitrile of formula: 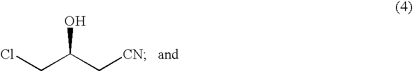
2a) dissolving 4-chloro-3-hydroxybutyronitrile of formula (4) in an alcoholic solvent, and then, reacting it with hydrogen chloride, or
2b) reacting 4-chloro-3-hydroxybutyronitrile of formula (4) in an alcoholic solvent saturated with hydrogen chloride, to form the 4-chloro-3-hydroxybutanoic acid ester of formula (1).

4. The process of claim 1, wherein the pH is adjusted in the range of 7.3 to 7.8.

5. The process of claim 1, wherein the pH is adjusted by adding an inorganic acid to the cyanide solution, and then, epichlorohydrin is added thereto.

6. The process of claim 5, wherein the inorganic acid is selected from the group consisting of hydrochloric acid, nitric acid, sulfuric acid, sulfonic acid, and phosphoric acid.

7. The process of claim 6, wherein the inorganic acid is sulfuric acid or concentrated hydrochloric acid.

8. The process of claim 1, wherein the cyanide is sodium cyanide or potassium cyanide.

9. The process of claim 2, wherein the alcoholic solvent is methanol or ethanol.

10. The process of claim 2, wherein the hydrogen chloride is anhydrous hydrogen chloride gas.

11. The process of claim 2, wherein the weight-by-weight ratio of the alcoholic solvent to 4-chloro-3-hydroxybutyronitrile is in the range of 1.5:1 to 2.5:1.

12. The process of claim 1, wherein epichlorohydrin or 4-hydroxybytyronitrile has optical activity.

13. The process of claim 3, wherein the pH is adjusted in the range of 7.3 to 7.8.

14. The process of claim 3, wherein the pH is adjusted by adding an inorganic acid to the cyanide solution, and then, epichlorohydrin is added thereto.

15. The process of claim 3, wherein the cyanide is sodium cyanide or potassium cyanide.

16. The process of claim 3, wherein the alcoholic solvent is methanol or ethanol.

17. The process of claim 3, wherein the hydrogen chloride is anhydrous hydrogen chloride gas.

18. The process of claim 3, wherein the weight-by-weight ratio of the alcoholic solvent to 4-chloro-3-hydroxybutyronitrile is in the range of 1.5:1 to 2.5:1.

19. The process of claim 2, wherein epichlorohydrin or 4-hydroxybytyronitrile has optical activity.

20. The process of claim 3, wherein epichlorohydrin or 4-hydroxybytyronitrile has optical activity.
